(19) United States
(12) Patent Application Publication
(10) Pub. No.: US 2011/0269648 A1
(43) Pub. Date: Nov. 3, 2011

(54) ELECTROCHEMICAL SENSOR SYSTEM

(76) Inventor: ANNE M. SCHWARTZ, Tualatin, OR (US)

(21) Appl. No.: 13/099,333

(22) Filed: May 2, 2011

Related U.S. Application Data
(60) Provisional application No. 61/330,397, filed on May 2, 2010.
(60) Provisional application No. 61/447,697, filed on Feb. 28, 2011.

(51) Int. Cl. C40B 60/12; C12Q 1/25; G01N 27/416

(52) U.S. Cl. 506/39; 204/400; 204/415; 204/403.01; 204/403.14; 204/406

(57) ABSTRACT

A electrochemical sensor system is provided. An example system utilizes electrical and steric properties of contaminants, such as pesticides, herbicides, and heavy metals to measure an ongoing concentration of multiple contaminants simultaneously in real time. An example system has a sensor array including sensors tuned to specific contaminants, each sensor having at least two conducting elements arranged in a capacitive relationship, for example, on a printed circuit board. A binding layer on the conducing elements of each sensor selectively binds a specific contaminant, which produces a signature change in a measureable electrical property, such as impedance. Enclosed sensors and chemical buffers preserve the chemical and physical environment of the contaminants for ongoing real-time measurement of dynamic concentrations. A delivery system enables samples containing contaminants to be automatically delivered to the array of sensors without adulterating the natural state of the samples.

### RELATED APPLICATIONS

This patent application claims the benefit of priority to U.S. Provisional Patent Application No. 61/330,397 filed May 2, 2010 and incorporated herein by reference, and to U.S. Provisional Patent Application No. 61/447,697 filed Feb. 28, 2011 and incorporated herein by reference.

### STATEMENT REGARDING FEDERALLY SPONSORED RESEARCH

The invention was made under a contract with an agency of the United States Government, the Office of NIEH Research, under grant number 1R43 ES018132.

### BACKGROUND

Recent research has demonstrated that chronic ingestion of pesticides, especially organo-pesticides, correlates with children and adults having developmental disabilities. Real-time measurement of pesticides has not previously been possible, but is needed by food manufacturers and water quality systems, to enable adjustment of the systems to eliminate or reduce the amount of pesticides and other contaminants, such as heavy metals, in food and water to protect human health.

High performance liquid chromatography (HPLC) and mass spectroscopy have been the gold standard for environmental assays, and ELISA, enzyme-linked immunosorbent assay, has also been used but is problematic. ELISA is a biochemical technique used mainly in immunology to detect the presence of an antibody or an antigen in a sample. The ELISA has been used as a diagnostic tool in medicine and plant pathology, as well as a quality-control check in various industries. In simple terms, in ELISA, an unknown amount of antigen is affixed to a surface, and then a specific antibody is applied over the surface so that it can bind to the antigen. This antibody is linked to an enzyme, and in the final step a substance (e.g., a label) is added that the enzyme can convert to some detectable signal, most commonly a color change in a chemical substrate.

Some disadvantages of these conventional techniques are very high cost and lack of portability due to size. Other problems are the chemical requirements and their maintenance, intolerance to harsh environments, requirement for trained personnel, time per assay (>24 hours to 2 weeks depending on the measurement), and so forth.

### BRIEF DESCRIPTION OF THE DRAWINGS

FIGS. 1-4 are diagrams of example conducting elements of sensors for detecting and measuring contaminants.

FIG. 5 is a diagram of an example charge transfer between a molecule and a conducting platform.

FIG. 6 is a diagram of an example working electrode.

FIG. 7 is a diagram of a working electrode with multiple wells on one metal conductor.

FIGS. 8-10 are diagrams relating sensor conductor configuration and geometry to specific contaminants and functionalities.

FIGS. 11-12 are diagrams of sensor strips having multiple sensors, each sensor targeting a different contaminant, or redundantly measuring a same contaminant for reproducibility.

FIG. 13 is a diagram of an example enclosure or chamber enclosing conducting elements of a sensor.

FIG. 14 is a diagram of example values of Malathion measured by the example system.

FIGS. 15-18 are diagrams of atrazine measurement results using the described sensor systems.

FIG. 19 is a diagram supporting expression and purification of the merP protein.

FIG. 20 is a diagram of example potentiostat schemas.

FIG. 21 is a diagram of an example sensor and manifold as well as an example assembled system, having ten multiplexor daughter boards connected to a mother board with and controlled with a pick; ten piezoelectric pumps connected to ten daughter boards then connected to a pick to control the fluid delivered to and from the sensor with four valves connected to a pick to control choice of fluids to the sensor.

FIG. 22 is a flow diagram of an example method identifying analytes on a nanoporous implementation of the example system.

### DETAILED DESCRIPTION

An electrochemical sensor system is provided. An example system utilizes electrical and steric properties of contaminants, such as pesticides, herbicides, and heavy metals to measure an ongoing concentration of multiple contaminants simultaneously in real time. An example system has a sensor array including sensors tuned to specific contaminants, each sensor having at least two conducting elements arranged in a capacitive relationship, for example, on a printed circuit board. A binding layer on the conducing elements of each sensor selectively binds a specific contaminant, which produces a signature change in a measureable electrical property, such as impedance. Enclosed sensors and chemical buffers preserve the chemical and physical environment of the contaminants for ongoing real-time measurement of dynamic concentrations. A delivery system enables samples containing contaminants to be automatically delivered to the array of sensors without adulterating the natural state of the samples.

The electrochemical sensor system leverages multi-scale design and in one implementation can integrate nanoporous membranes attached to a microelectronic platform to generate a miniaturized multi-well plate similar to the ELISA plate. The system provides a-label free biomolecule detection technology and a real-time automated remote device. The system provides enhanced sensitivity with ELISA-comparable specificity.

In one implementation, the system incorporates techniques of miniaturization, automation, packaging and assembly in producing the scalable, automated real-time platform. In one implementation, the system uses a printed circuit board (PCB) sensing platform, which provides the advantages of inexpensiveness and robust design. Inter-digitated electrodes offer enhanced surface area for increases sensitivity.

A nanoporous membrane coating or layer on the sensor conductor elements may be used to create an array of nanowells, similar to ELISA. Alumina or multi-walled carbon nanotube (MWCT) membranes may be used in one implementation. These provide the advantage of label-free technology replicating the benefits of ELISA, but on an inexpensive and robust electrical platform, instead of an optical platform requiring chemical labels to provide optical markers.

In one implementation, the system has at least one sensor calibrated for atrazine binding to anti-atrazine antibody. Anti-atrazine is used as the antibody binding layer for the conducting element of a sensor, which then recognizes all morphologies of atrazine. Anti-chlorpyrifos may be used as a nonspecific control. In one implementation, anti-atrazine antibody is highly specific for atrazine with less than 15% of nonspecific binding. Nonspecific binding may result from binding to surfaces other than antibodies as a result of the substrate used to adhere the antibody to the sensor. A blocking agent may be successfully used to prevent nonspecific binding.

In one implementation, interdigitated patterns for the conducting elements of the sensors on a printed circuit board platform enable a low cost (e.g., $0.11 USD per sensor), and the sensors can be recycled and use repeatedly.

A method of automatically delivering a sample to a sensor or sensor array provides real-time monitoring of multiple contaminants, with a processor or controller operating the system and reading the results via a USB connection to a computer.

In one implementation, an anodisc alumina membrane with 200 nm pore size can be overlaid on the sensing site. The addition of membranes creates a well structured array of nanowells mimicking ELISA technology.

A Teflon encapsulation chamber may enclosed the sensing site. This chamber can eliminate signal variability and protein denaturing due to evaporation issues.

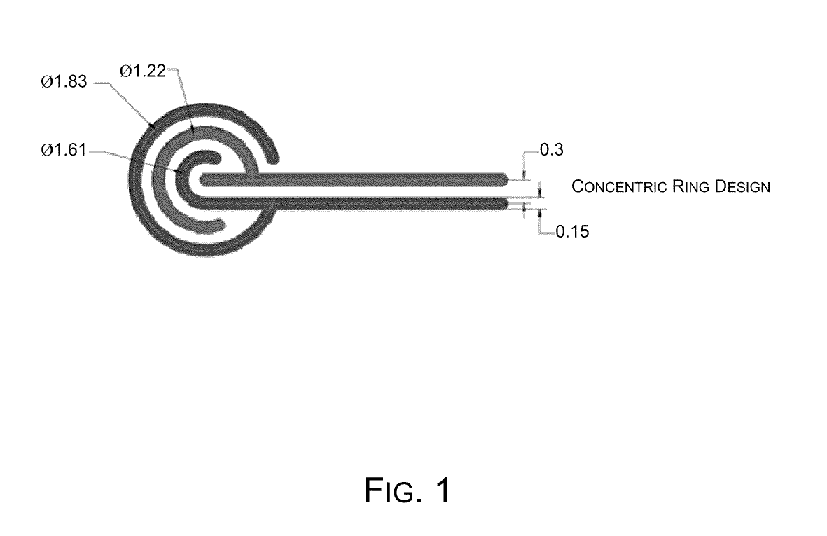

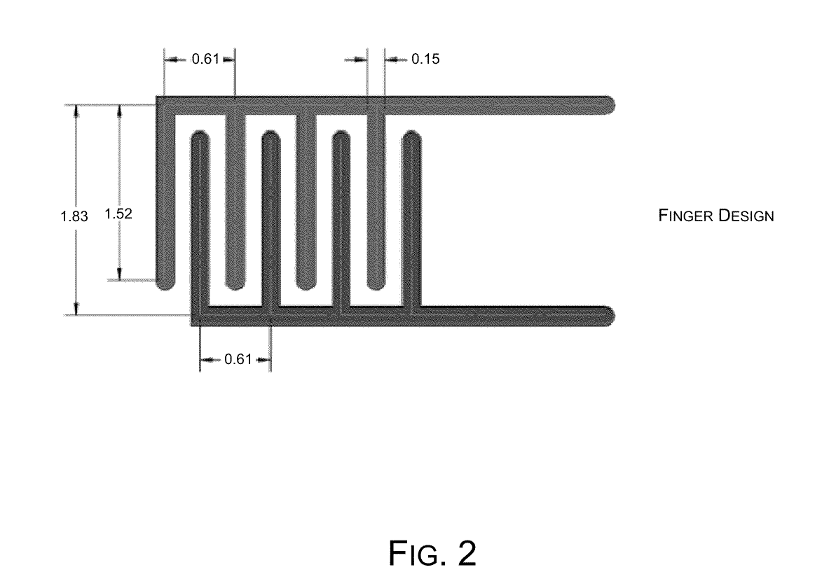

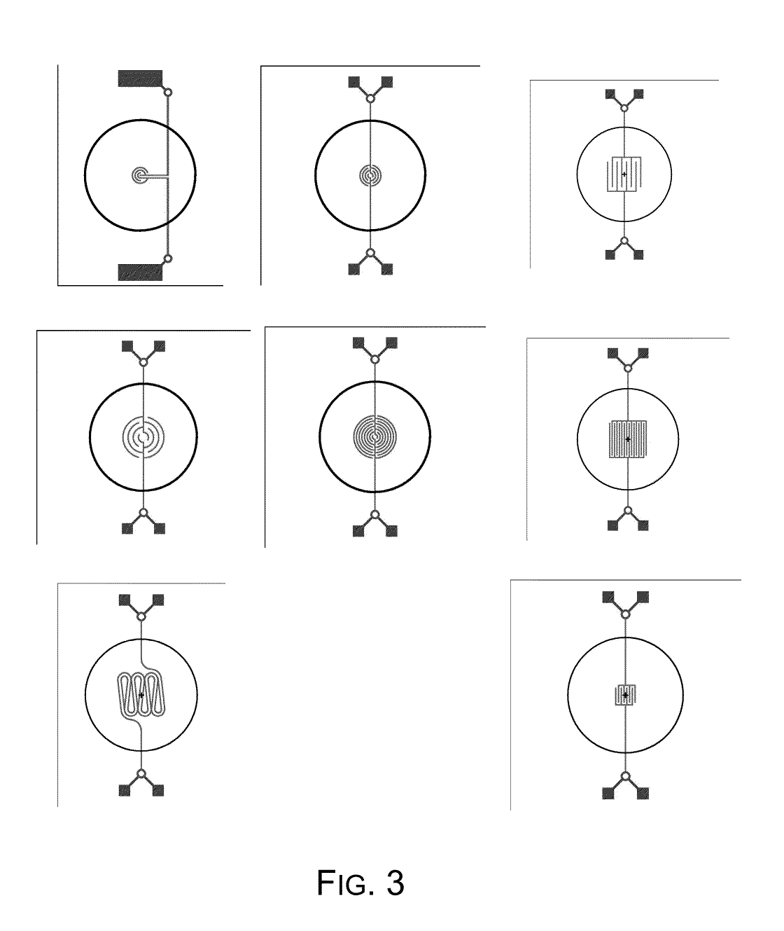

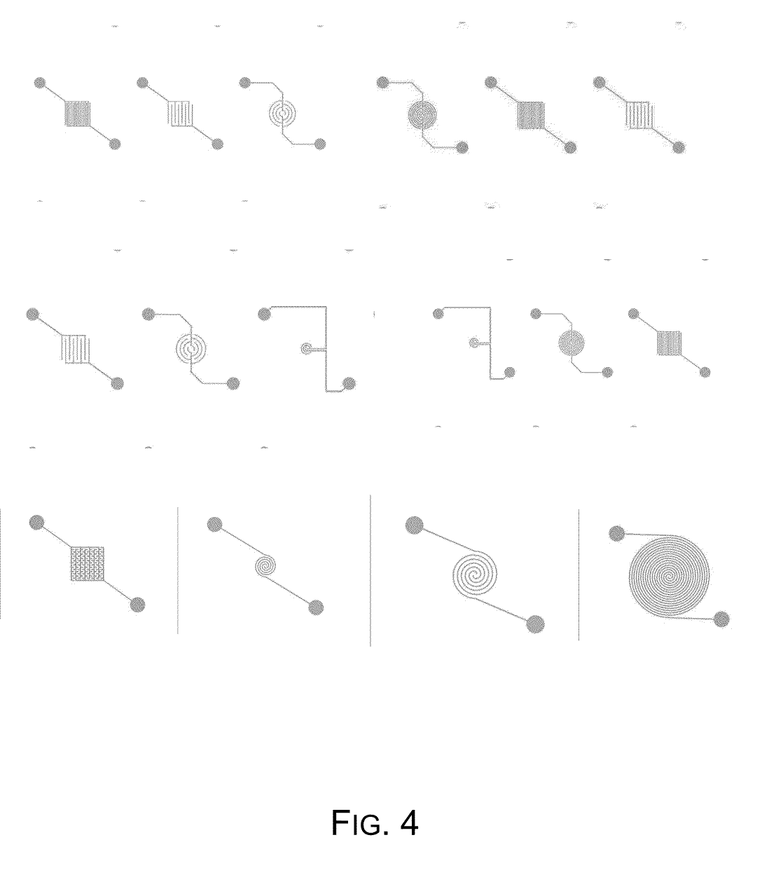

In one implementation, the electrochemical sensor system for measuring contaminants has sensors that include two conductors arranged in a capacitive relationship on a printed circuit board, with various configurations and geometries as shown in FIGS. 1-4. In one implementation, the capacitance at play and giving rise to an impedance signature for the contaminant at hand is given by Equation (1):

$$\frac{1}{{C}_{tot}} = \frac{1}{{C}_{sub}} + \frac{1}{{C}_{sl}} + \frac{1}{{C}_{lAb}} + \frac{1}{{C}_{Ab - Ag}} \qquad Equation ( 1 )$$

where
Cₜₒₜ: Total Capacitance
C_{sub}: Substrate Capacitance
Cₛₗ: Linker Capacitance
C_{lab}: Antibody Capacitance
C_{Ab-Ag}: Antibody-Antigen Binding Complex Capacitance

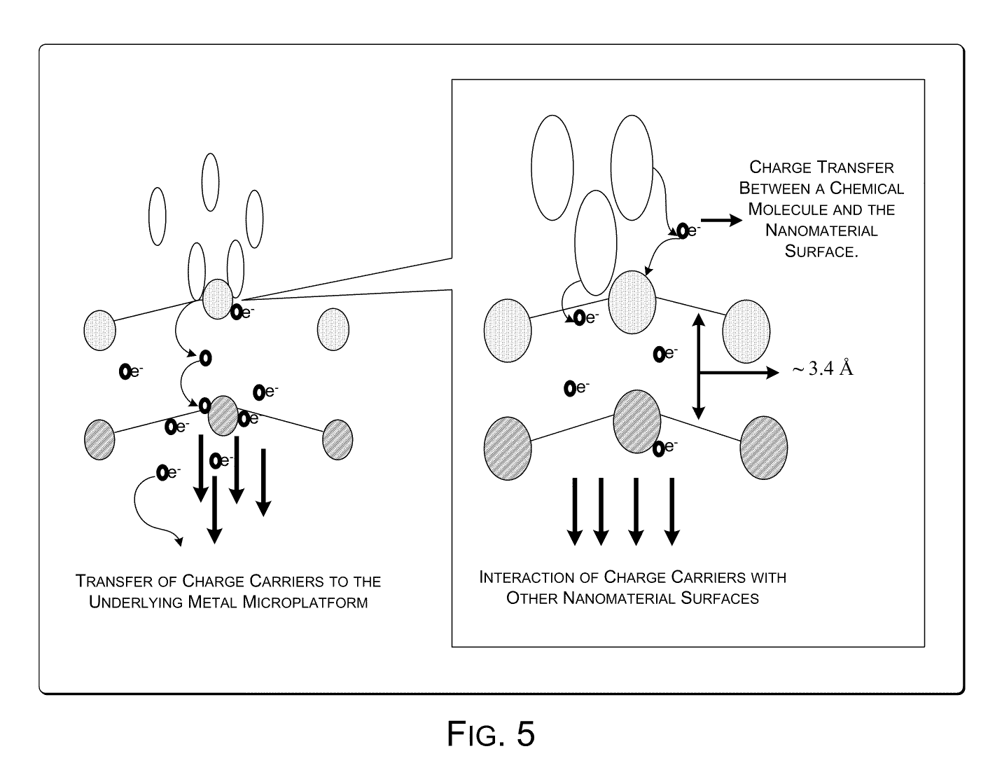

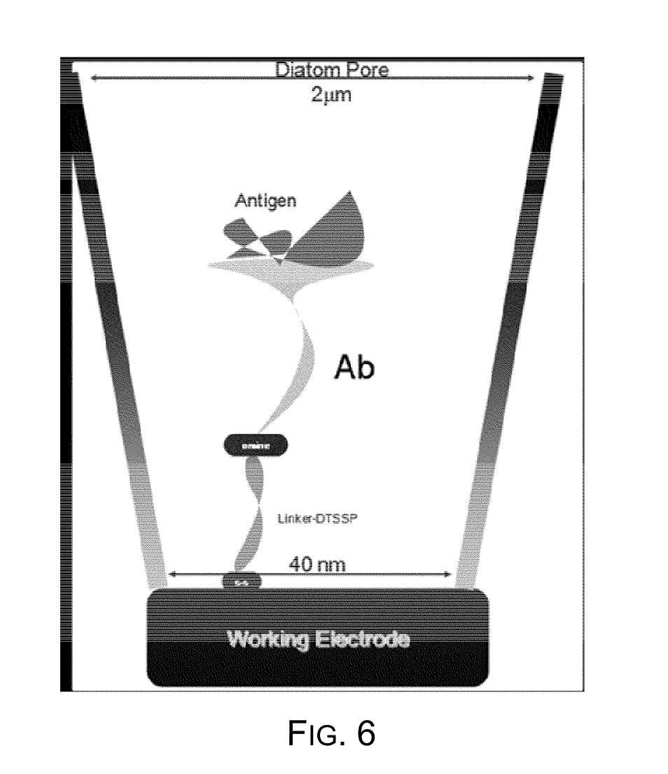

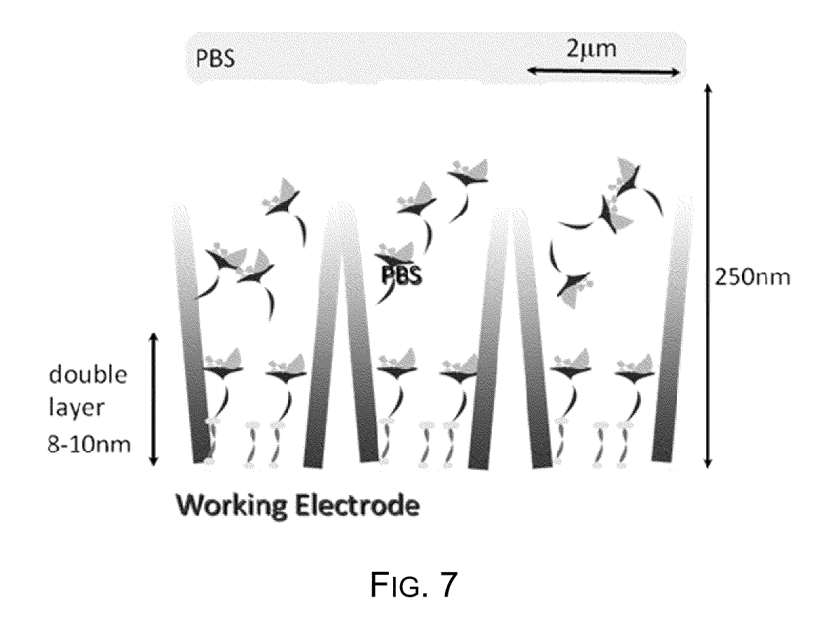

In one implementation, as shown in FIGS. 5-7, the measurement technique is based on determining an impedance originating from the real-time capacitance (e.g., dielectric and/or electrical bilayer properties) at the interface of the nanowell. The formation of the sandwich assay results in the double layer at the nanowell interface between a selective binding layer attached to conducting elements of a sensor and the contaminant bound. In one implementation, the addition of the antibody and antigen in a serial method similar to ELISA results in the protein specific changes to the measured capacitance. Multiple nanowells on the single sensing site, as shown in FIG. 7, result in signal averaging, which reduces the variability in the measurement.

A sensing module connected to the two conductors forms an electrical circuit and measures a change in impedance resulting from an amount of the particular contaminant currently bound to the binding layer. An enclosure (chamber) around at least part of the two conductors maintains an environment of the particular contaminant for real time measurement of the particular contaminant.

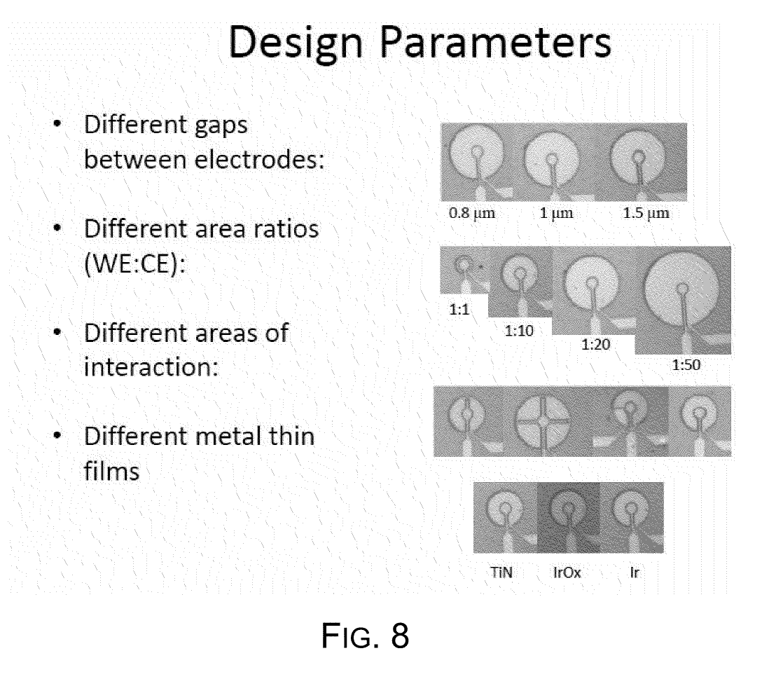

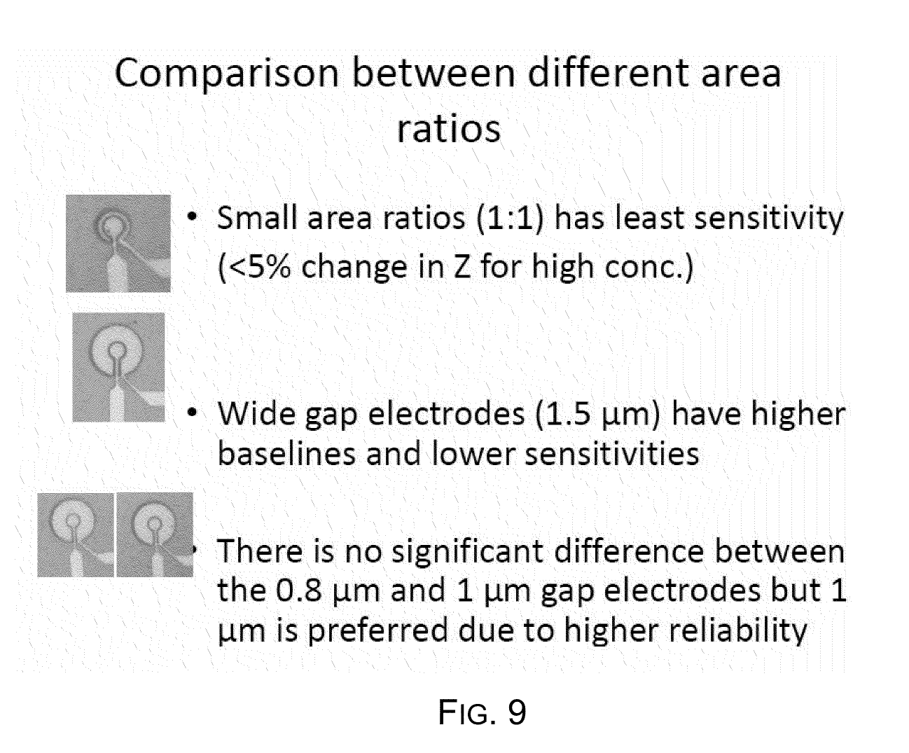

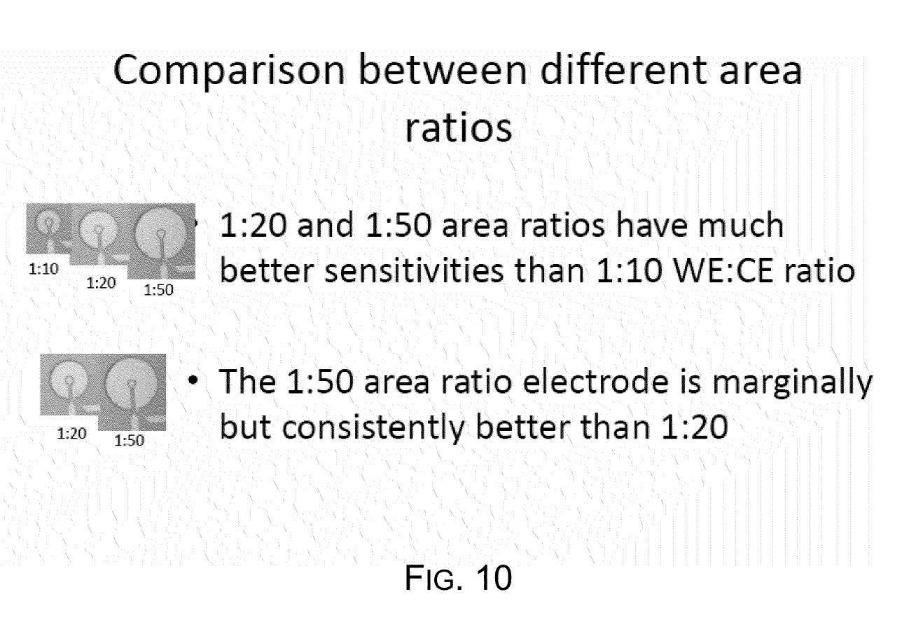

As shown in FIGS. 8-10, in addition to a binding layer being tuned to bind a particular contaminant to the sensor for measurement, the capacitive relationship of the two conductors itself may be tuned to the particular contaminant. The capacitive relationship may be tuned to a particular contaminant by varying a pattern or configuration of the two conductors printed on the printed circuit board, distance between conductors (i.e., analogous to distance between capacitor “plates”) a physical characteristic of a material composing at least one of the conductors, by doping of at least one of the conductors, varying the length of the conductors on which the contaminant is senses, and so forth.

The size of a surface area of a pattern of the conductors printed on the printed circuit board may be selected to provide a sensitivity range for sensing the particular contaminant.

The sensing module may be configured to measure an impedance indicative of dielectric changes in the binding layer caused by an amount of the particular contaminant currently bound to the binding layer on at least one of the two conductors.

A nanoporous layer between a conducting surface of at least one of the two conductors and the corresponding binding layer can increase a detection sensitivity of the sensor, e.g., when the nanoporous layer is an alumina membrane, a carbon nanotube (CNT) membrane, or a multi-wall carbon nanotube (MWCT) membrane.

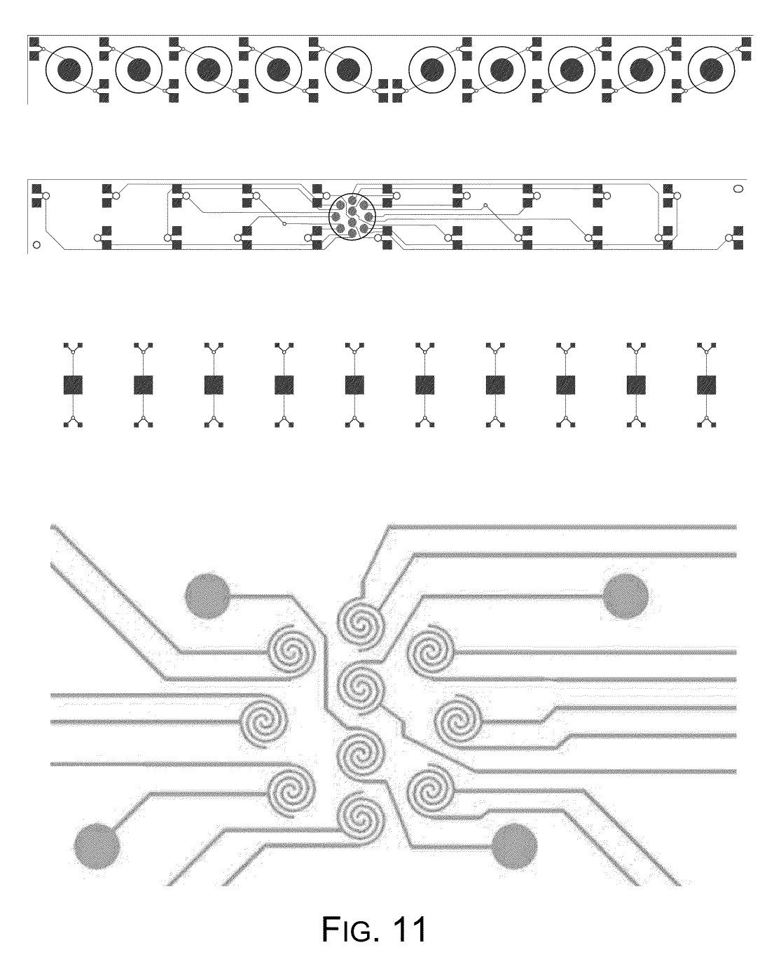

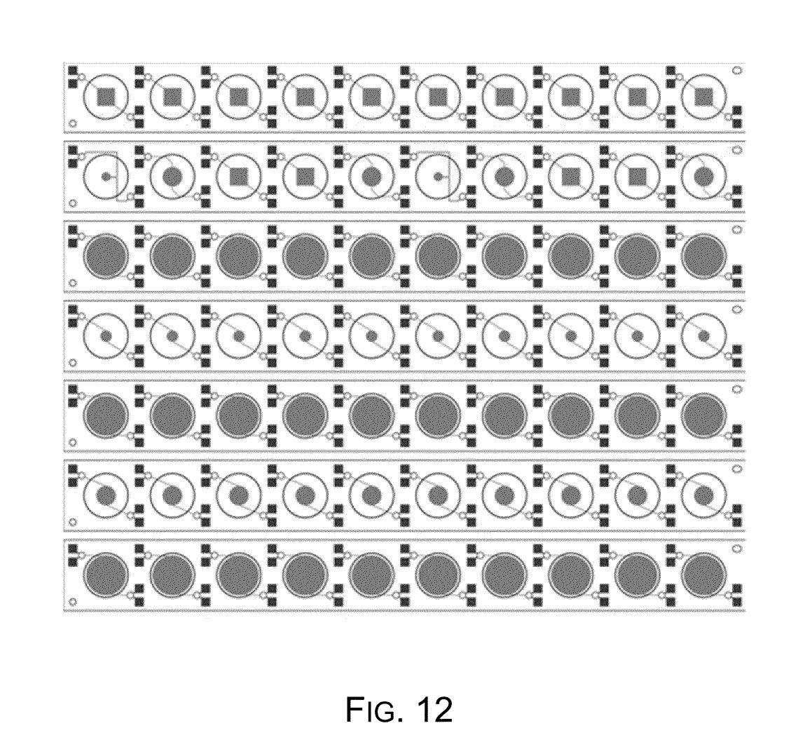

As shown in FIGS. 11-12, an example system has a sensing module that performs multiplex detection of multiple contaminants, using strips of multiple individual sensors, i.e., arrays of a mix of sensors, each sensor tuned to a different contaminant. Contaminants may have an impedance signature, or the sensor may be specific for the particular contaminant so that a gross impedance measurement varies with concentration of the contaminant. In one implementation, the sensing module probes a frequency response of a contaminant mixture and compares obtained frequency responses against a library of frequency response signatures of individual contaminants.

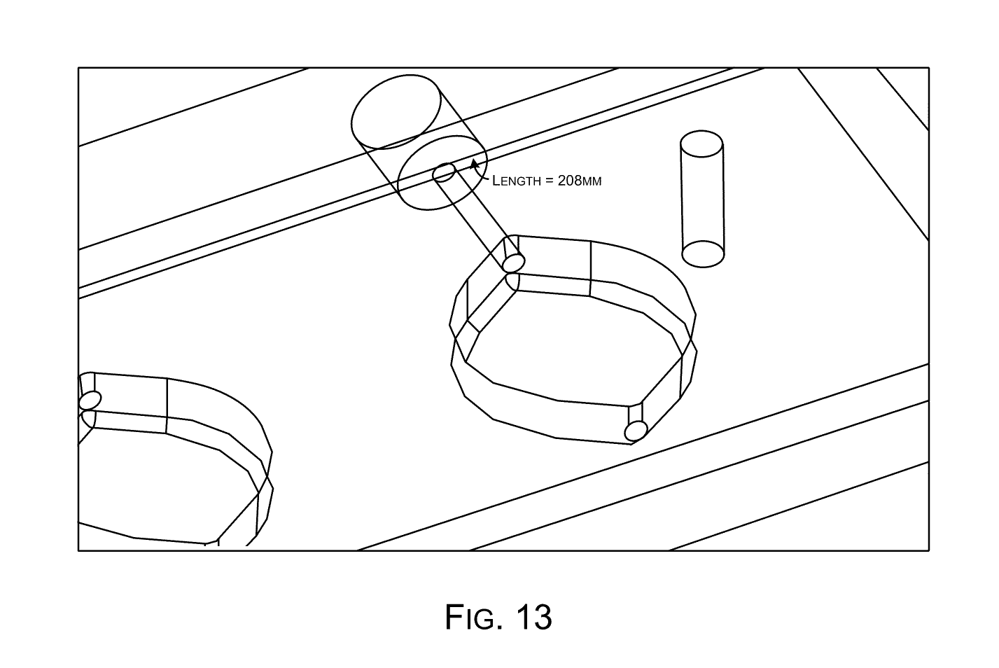

One or more buffer solutions maintain the environment of the contaminants for measurement and/or maintain a state or a surface of the sensor or binding layer. Buffer solutions may include a solubilization buffer, an immobilization buffer, a wash buffer and a linking buffer, these are used in the enclosure (chamber) as shown in FIG. 13, around at least part of the two conductors to maintains an environment of the particular contaminant for real time measurement of the particular contaminant.

The electrochemical sensor system may thus provide real-time dynamic measurement of multiple contaminants to be sensed by an array of sensors, the contaminants including, pesticides, herbicides, proteins, heavy metals, antibodies, drugs, and bacteria. The pesticides may include malathion and chlorpyrifos; the herbicides may include atrazine; and the heavy metals may include mercury and lead.

A selection of a binding layer specific to a particular contaminant to be attached to the conducting elements of a sensor may be one of the following diverse list of binding layer candidates, although the list is not exclusive or comprehensive:

a mercury binding protein;

a heavy metal binding protein;

an anti-atrazine antibody;

an anti-malathion antibody;

an anti-chlorpyrifos antibody;

an enzyme;

an oligosaccharide;

a nucleotide or oligonucleotide;

a cholinesterase enzyme;

a pesticide binding protein;

a metallo-enzyme;

a cell receptor protein;

a peptide;

a lipid;

a protein;

a drug;

a drug target;

a neurotransmitter;

a carboxyl group;

a herbicide binding protein;

a fungicide binding protein;

a biomarker binding protein;

a DNA binding protein; or

a DNA motif.

The sensing module creates a circuit between the conducting elements of a sensor tuned to a particular contaminant and may include a pulse generator to send an electrical pulse across the two conductors. The electrical pulse can be a voltage waveform or an electrical current waveform. The electrical pulse is tailored to detect an impedance signature of a specific contaminant.

The binding layer binds the particular contaminant reversibly and the monitor provides ongoing real-time measurement of a dynamic concentration of the particular contaminant in a gas, a liquid (e.g., air or water sample). The electrochemical sensor system can thus have an array of sensors, each sensor tuned to a different contaminant, provide ongoing real time measurement of dynamic concentrations of multiple contaminants in one of a gas, a liquid, air, or a water sample.

The electrochemical sensor system can include a laser processing unit for sample preparation. In one implementation, the system is configured to draw a sample from a food processing line directly or indirectly, and the food sample is processed by dilution in fluid, by being dissolved in fluid, digested, bead blundered, or vaporized and dissolved in fluid and then sent to the sample fluid delivery system for deposition on a sensor.

The electrochemical sensor system may have hardware and software programmed to receive, e.g., analog information based on the electrical signals from the sensing module and translate this information into database entries. The system may print the name and amount of contaminants found in a food sample on a tag, barcode or label to be attached or printed on to a food product.

The system optimizes electrical design of devices and sensors, including minimized conversion of surface charge to capacitance. Buffers and blockers reduce nonspecific binding and increased antibody binding to the pesticide. Chamber materials may use Teflon and stainless steel to reduce loss of pesticide caused by adhering to storage containers and mechanical parts.

| Characteristic Feature | ELISA | PCB biosensor device |
|---|---|---|
| Method of Detection | Labeled optical detection | Label - free detection |
| Portability | Not portable | Portable |
| Detection | Lab based (skilled | Point of care (non |
| | personnel) | specialist personnel) |
| Dynamic range | Three orders of | At least 6 orders of |
| | magnitude | magnitude |
| Limit of Detection | Pico-molar regime | Nano - molar regime |

The exemplary system has advantages over the existing techniques, namely speed, minimal training of operating personnel and low cost. The sensing technique is label free, hence no external chemicals or tags are needed and thus the risk of contamination is reduced. The system can be made into a portable real-time remote device.

Select Binding Layers

A binding layer may be selected and attached to the conducting elements of a sensor in order to selectively bind a desired contaminant. A list of candidate binding layer proteins, etc., is given above. For example, a mercury binding protein is appropriate for selectively measuring concentrations of mercury in real time. While a pesticide binding layer selectively forms ligands or otherwise binds to select pesticides.

Malathion is an organophosphate parasympathomimetic which binds irreversibly to cholinesterase. Malathion is an insecticide of relatively low human toxicity, however recent studies have shown that children with higher levels of malathion in their urine seem to be at an increased risk of attention deficit hyperactivity disorder.

Chlorpyrifos (IUPAC name: O,O-diethyl O-3,5,6-trichloropyridin-2-yl phosphorothioate) is a crystalline organophosphate insecticide that inhibits acetylcholinesterase and is used to control insect pests. Chlorpyrifos is moderately toxic and chronic exposure has been linked to neurological effects, developmental disorders, and autoimmune disorders.

Example Pesticide Binding Layer

In one implementation, an antibodies are utilized in the binding layer, for example, anti-atrazine (lifespan biosciences catalog # LS-C74423) or anti-chlorpyrifos, anti-malathion, can be used as the active ingredient in the binding layer. An anti-atrazine implementation of the binding layer samples consisting of 1 mg/ml solutions in a methanol buffer were diluted directly in water after calculating the appropriate ratios. Specific interaction of atrazine with the antigen binding layer yielded impedance change up to 32% in relation to concentration of the atrazine. Nonspecific interactions also generate an impedance change of 1%-5%, but the limit of detection can be as high as 500 ppt (parts per trillion).

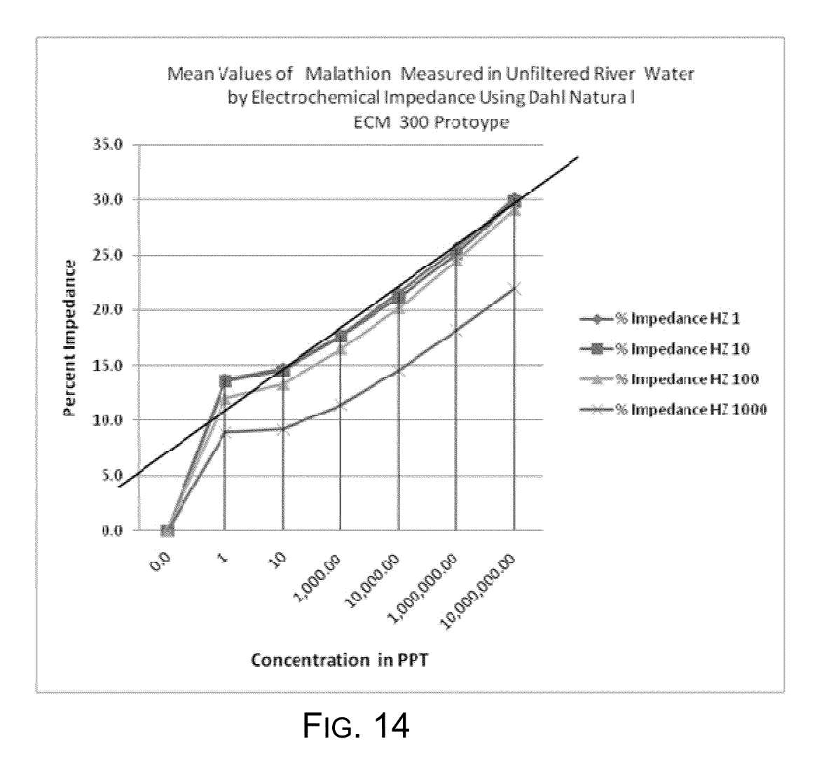

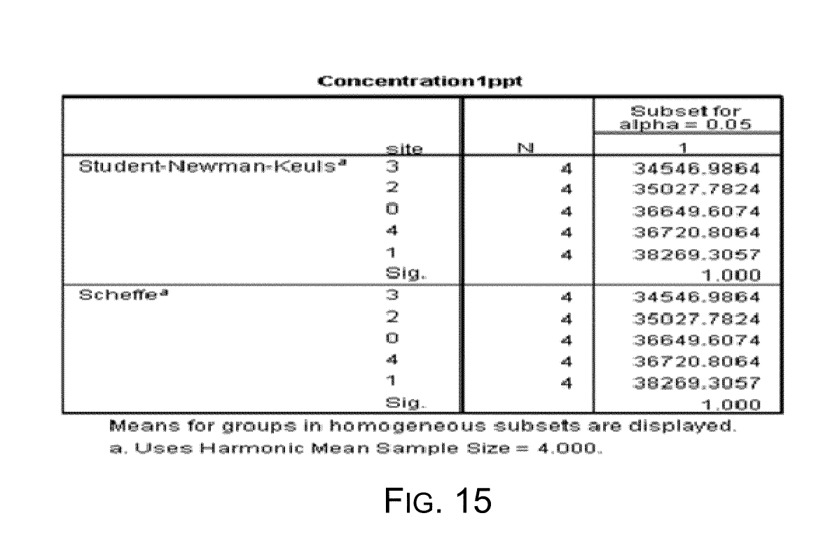

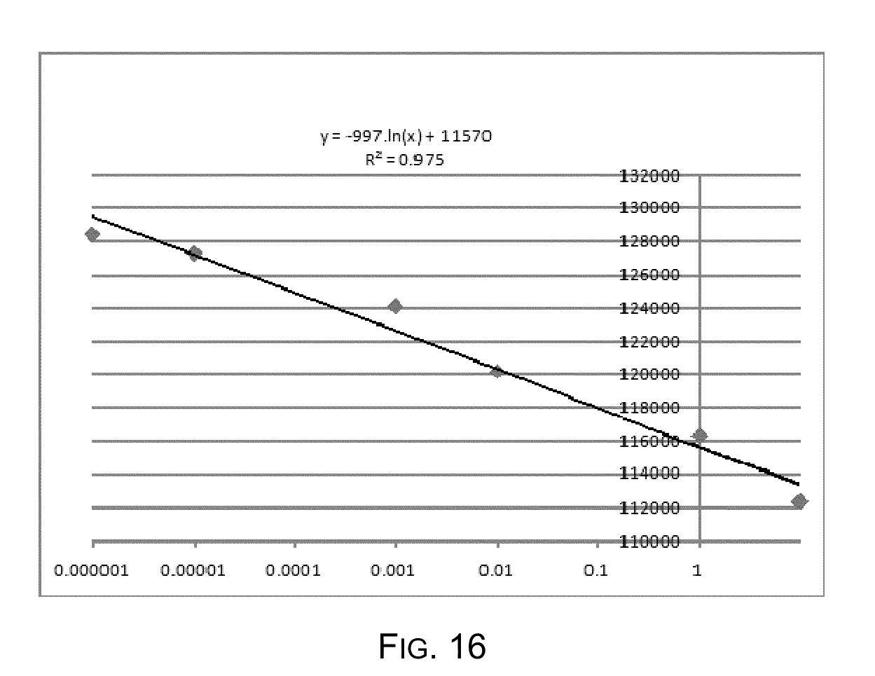

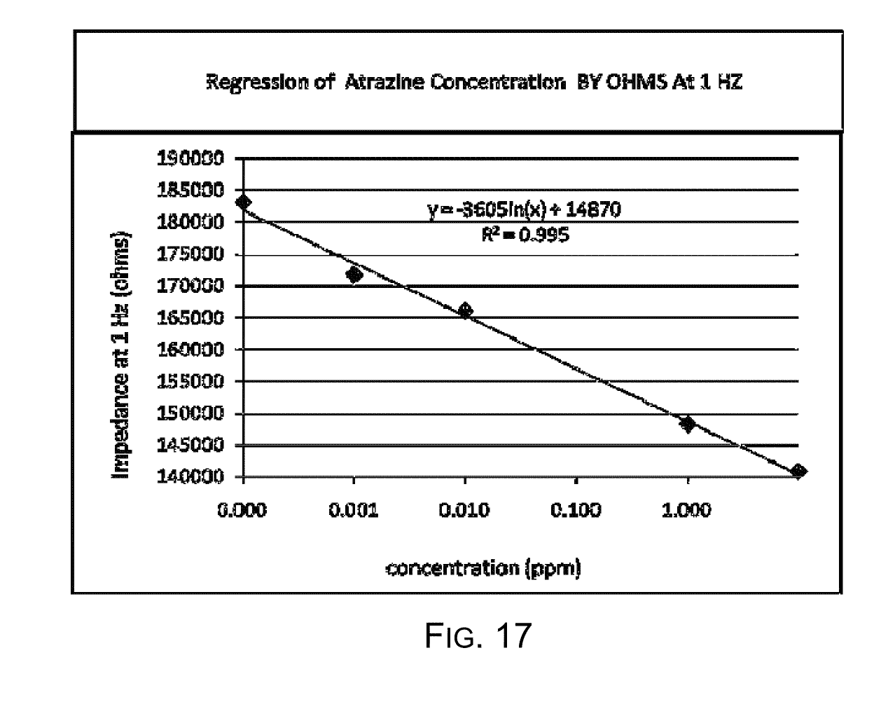

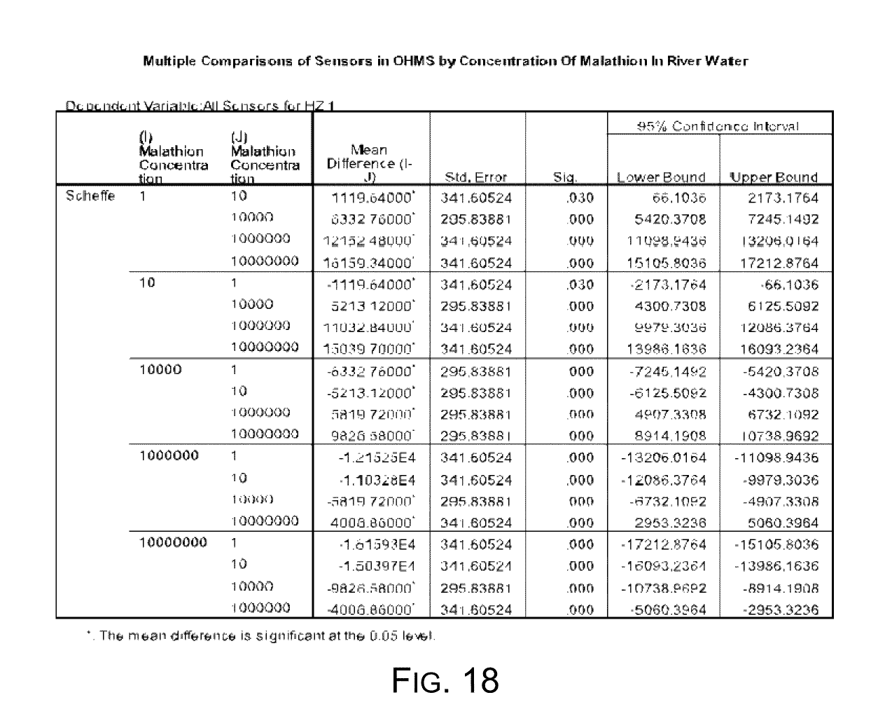

FIGS. 14-18 show results of an example system implementing impedance measurement of concentrations of the pesticide Malathion with sensors selective for atrazine.

Example Mercury Binding Layer

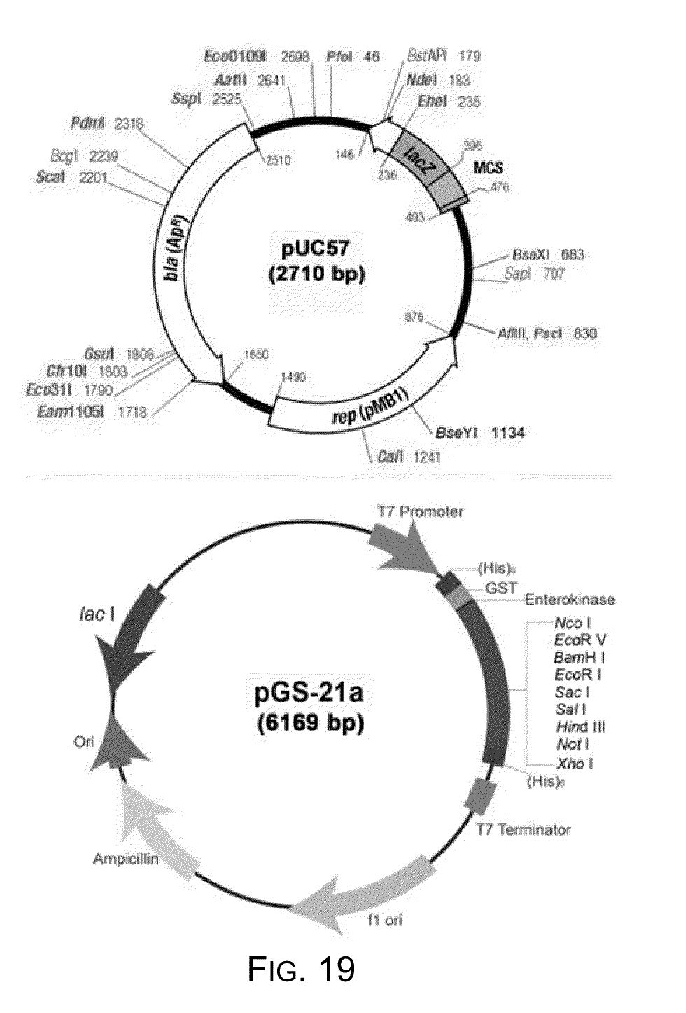

In another implementation, a mercury binding protein, such as merP is selected as the binding layer. Referring to FIG. 19, expression and purification of the merP protein is now described. Certain microorganisms such as bacteria thrive in environments that contain a heavy amount of heavy metals since they have evolved efficient mechanisms for detoxification of these toxic metals. The bacterial mercury detoxification system is remarkable. It functions by transporting toxic Hg(II)—the organic form—into the cell where it is converted to relatively nontoxic metallic Hg(0) which is volatile and can be passively eliminated The sequences of the proteins responsible for mercury detoxification are encoded in the mer operon on a plasmid that typically also has operons that confer antibiotic resistance. Among these proteins, the merP protein binds mercury in the periplasm and transfers it to the mercury transport protein responsible for transporting mercury through the membrane into the cytoplasm. Like other metal binding proteins, MerP contains the Cys-X-X-Cys motif and coordination to the cysteines is the dominant metal binding mechanism. The full length protein of Shigella flexneri is 91 amino acids long. This is processed further into a mature form which is 72 amino acids. Within the sequence, the 18 residue fragment Thr-Leu-Ala-Val-Pro-Gly-Met-Thr-Cys-Ala-Ala-Cys-Pro-Ile-Thr-Val-Lys-Lys forms the metal binding loop. However a sensor using this 18 amino acid peptide fragment as the mercury binding ligand showed poor specificity to mercury. Ions like zinc, cadmium and sliver ions bound showed greater affinity to this peptide on the sensor than mercury itself. Hence we have devised a strategy to express the full length mature protein, which is well known to have excellent specificity and affinity to mercury, for use on the sensor. The other protein may be of use in nonspecific applications for screening.

The nucleotide sequence of the native merP gene was modified to so that the codons were optimum for protein expression in E. coli. A 6×HIS tag was added to the C-terminal of the protein to facilitate purification of the protein. Flanking this sequence, two restriction sites were also added to enable subcloning into a protein expression vector. The cloning vector used was pUC57. From here the modified merP gene was subcloned into the E. coli expression vector pGS21a. This vector has a IPTG inducible T7 promoter and can be used for high-level protein expression in E. coli.

Both the plasmids were first propagated in DB3.1 strain of the E. coli cells. Plasmids extracted from DB3.1 cells were run on an agarose gel along with a 1 kb ladder. Image of the Ethidium bromide stained gel image captured under a UV transilluminator is shown here. The pUC57 plasmid runs lower than the pGS21a due to its smaller size.

Next, pGS21a plasmid was used for transformation of the protein expression host BL21*. BL21* E. coli strains are high-performance BL21 hosts designed for improving protein yield in a T7 promoter-based expression system. Because T7 RNA polymerase synthesizes mRNA more rapidly than E. coli RNA polymerases, transcription from the T7 promoter is uncoupled to translation in E. coli. This results in mRNA transcripts unprotected by ribosomes, which are then subject to enzymatic degradation by endogenous RNases. The reduced level of transcripts in the cell often leads to greatly reduced levels of protein yield. The BL21 Star™ strains contain a mutation in the gene encoding RNaseE (rne131), which is one of the major sources of this mRNA degradation (2). BL21 Star™ cells significantly improve the stability of mRNA transcripts and increase protein expression yield from T7 promoter-based vectors (3).

After transforming the BL21* cells, transformed cells were screened by antibiotic selection on LB agar plates. Currently, selected BL21* cells containing the pGS21a-merP plasmid are being cultured so that they can be induced for merP production and protein purification.

Next, this was followed by purification of the protein and characterization of protein quality and quantity.

Example DNA Binding Layer

For some applications a sensor layered with DNA binding protein has advantages over an antibody type binding. Some advantages as compared to antibody-based diagnostic kits are: 1) a limited shelf life, 2) greater instability due to sensitivity to higher temperatures and/or moisture. Our sensor platform as previously described uses a target specific DNA-binding protein(s) to bind unique diagnostic DNA targets of interest. The binding events are detected through electrochemical means. The fact that this is possible without optical detection requiring the use of florescent labels provides greater reliability and flexibility of targets since all targets may not be measured accurately by use of a label.

An Example of this type of measurement is to use a commercially available and purified DNA-binding protein (human estrogen receptor), and to measure its specific interactions with the corresponding DNA motif (=estrogen response element) which can be supplied in the form of a short synthetic piece of double stranded DNA representing the binding sequence of estrogen. To date, the genomes of many medically relevant diseases, bacterial and parasite targets for diagnosis of illness have been completely sequenced, and the DNA data is available to the public. However libraries of DNA motifs capable of identifying the biomarker, bacteria, parasite or disease need to be created. This is very time consuming since many segments (motifs) of the DNA must be screened. The present invention has the capability of using an array of 70 sensors composed of 10 sensing replicates built into each of seven arrays to electrochemically detect DNA binding of a single target ten times simultaneously (seventy 70). The computing software is then able to calibrate from a known sample blank the entire array and further statistically compare the ten replicates using reliability statistics since each sensor is capable of six repeated measures to validate a dose response curve (see bottom sensor element pattern in FIG. 11). So this sensing device, hardware and software would greatly conserve samples since for each of the ten grouped sensors a 200 micro liter sample would be applied to the entire array and then measured simultaneously then rinsed and increasingly dosed with samples seven times to create a dose response curve. The ability for replication within each sample provides a built in method of validity and reliability of the motif as well as dynamic range and sensitivity limitations of the system for each motif. In current systems using optical methods more sample is needed and there is greater variability in testing since dose response curves cannot be calculated on the original DNA binding protein because each dose creating a dose response curve would have to be done on a new sensor. Therefore this invention would greatly increase the ability to due high throughput screening of motifs to create libraries DNA targets/motifs that indicate disease/infection. In the field this method could be used to screen for many disease targets at once since many different DNA binding proteins could be loaded on each sensor. It is possible that entire blood panels could be tested at once using this system to improve diagnostic capabilities especially in low resource areas. Lastly this invention could greatly enhance access to clinical and environmental diagnostics for disease/infection in low resource areas using a portable model of the device.

Example Nanoporous Layer Embodiments

In one implementation, a nanoporous membrane can be applied to sensor conductor elements, upon which the binding layer is then applied.

Below is described various protocols for preparing and attaching carbon nanotubes to a sensor, which provide various benefits.

### CNT Protocol

### Sensor Volume=0.2 ml

Targets: 0.1 mg/ml of CNT per sensor and 0.010 mg/ml of Protein per sensor (conjugated to CNT)

Absolute mass on ONE sensor: CNT=(0.1 mg/ml)(0.2 ml)=0.02 mg

Protein=(0.01 g/ml)(0.2 ml)=0.002 mg

### 10 Sensor Prep:

(10 sensors)(0.2 ml)=2 ml====Extra 10%=2.2 ml volume needed for 10 sensors

### CNT

(0.1 mg/ml CNT/sensor)(0.2 ml/sensor)=0.02 mg CNT/sensor
(0.02 mg/sensor)(10 sensors)=0.2 mg CNT=====Extra 10%=0.22 mg

### Protein

(0.01 mg/ml protein/sensor)(0.2 ml/sensor)=0.002 mg protein/sensor
(0.002 mg/sensor)(10 sensors)=0.02 mg CNT=====Extra 10%=0.022 mg

### Buffers

Solubilization Buffer (SB)—10% Ethanol in water
Immobilization Buffer (IB)—50 mM mono- and di-basic phosphate
Wash Buffer (WB)—25 mM sodium phosphate buffer
Linking Buffer (LB)—0.5% glutaraldehyde in IB (25 mM sodium phosphate buffer, pH 7.0)

### Recommendations:

Sonication—0.5 hours to several hours
CNT solubility—10 to 100s of ug/ml
Concentration on sensor—100s of ug/ml (in 0.2 ml)
Centrifuge—>5000 g, time to be determined
Solvent for CNTs—start at 10% ethanol in water
Method 1—Attach CNT to DSP on Sensor then Immobilize and Conjugate Protein to CNT.

### Solublization

Suspend CNTs in SB at a concentration of _{—}0.25_ mg/ml and sonicate for _{—}1.0_ hours.
10 ml Prep=>10 ml SB+2.5 mg CNT (need large volume to have measureable amount of CNT)
Sonicate in 15 ml Falcon tube

### Link to DSP on Sensor

Dilute 0.25 mg/ml CNT of solublized CNT to 0.1 mg/ml with a volume of 2.2 ml

(0.25 mg/ml)V=(0.1 mg/ml)(2.2 ml)
V=0.88 ml+2.112 ml of SB
   Add 0.2 ml of CNTs in SB at _{—}0.1_ mg/ml to each DSP modified well and incubate for _{—}1_ hour.
   Wash sensor with SB.
   Wash sensor with PBS.
   Make measurement.
   Adsorption of Protein onto CNT
   Wash sensor once with IB.
   Add 0.2 ml of protein at _{—}0.01_ mg/ml in 25 mM IB to the sensor.
   Incubate at room temperature for _{—}2_ hours.
   Cross-Linking of Adsorbed Protein with Glutaraldehyde
   Add _{—}0.2_ ml of 1% LB per well. (Added on top of IB, therefore the final concentration is half, or the desired 0.5% glutaraldehyde in 25 mM IB)
   Incubate for _{—}1_ hour.
   Wash with WB.
   Wash with PBS.
   Take measurement
   Continue on with Blank PBS then target.

### Time to Complete:

Setup (Gamry, Netbook, prep DSP and the chip)—0.75 hour
Sonicate—2 hour (Take baseline, incubate DSP on chip, and take reading of sensor here)
CNT-DSP binding, measurements—1.5 hours (1 hour incubation, 0.5 hour measurement)
Adsorption of protein and measurements—2.5 hours (2 hours incubation and 0.5 hour measurement)
Time to analyze 5 target concentrations (including Blank)—5 hours
Clean up, shut down, waste disposal—0.25
Total: 12 hours. Extra time to solublize, adsorb, or conjugate will extend this estimation.
Method 2—Cross-Linking of Proteins to CNT and then Link to Sensor with DSP

### Solublization

Suspend CNTs in SB at a concentration of _{—}0.25_ mg/ml and sonicate for _{—}1.0_ hours.
10 ml Prep of a 0.25 mg/ml solution=>10 ml SB+2.5 mg CNT (need large volume to have measureable amount of CNT)
Sonicate in 15 ml Falcon tube
Adsorption of Protein onto CNT
Mix _{—}0.88_ ml mg of CNT at _{—}0.25_ mg/ml (total of 0.22 mg CNT) with _{—}0.022_ ml of protein at 1 mg/ml (total of 0.022 mg Protein). Volume=0.935 ml, so add 0.935 ml of 2×IB (50 mM) for final concentration of 25 mM IB
Mix at room temp for _{—}2_ hour in glass bottle.
Transfer mixture to 1.5 ml microcentrifuge tube and centrifuge at _{—}10000_ g for _{—}10_ min then wash 2× with 1.0 ml of WB.
Cross-Linking of Adsorbed Protein with Glutaraldehyde
Reconstitute CNT-Protein with _{—}1.0_ ml of LB.
Mix for 1 hour at room temperature.
Centrifuge at _{—}10000_ g for _{—}10_ min then wash 3× with 1.0 ml of WB.
Reconstitute in 2.2 ml of PBS (which will then contain 0.1 mg/ml CNT with 0.01 mg/ml protein attached)

### Conjugation to Sensor

Take the 2.2 ml solution of CNT-Protein and add 0.2 ml to each DSP activated well
Incubate for 1 hour at RT.
Wash with PBS 2×
Take measurement.
Continue on with Blank PBS then target.

### Time to Complete:

Setup (Gamry, Netbook, DSP, chip)—0.75 hour
Sonicate—1 hour (Take baseline reading of sensor here)
Adsorption—2 hours (DSP incubation and readings in parallel)
Wash—0.5 hours
Cross-linking—1 hour
Wash—0.5 hours
On sensor DSP and CNT-protein cross-linking—1 hour.
Time to analyze 5 target concentrations (including Blank)—5 hours
Clean up, shut down, waste disposal—0.25
Total: 12 hours. Extra time to solublize, adsorb, or conjugate will extend this estimation.

### CNT Protocol (Bench for AFM)

For AFM analysis.
Linking Buffer 1 (LB1)—15% glutaraldehyde in 200 mM phosphate buffer, pH 7.0
Linking Buffer 2 (LB2)—0.5% glutaraldehyde in 25 mM sodium phosphate buffer, pH 7.0
Immobilization Buffer (IB)—25 mM sodium potassium
Wash Buffer (WB)—25 mM sodium phosphate buffer

### Solubilization Buffer (SB)—?

Method 1—Glutaraldehyde Activate CNTs then Attach Protein.

### Solublization

Suspend CNTs in SB at a concentration of ______ mg/ml and sonicate for ______ hours.

### Glutaraldehyde (GA) Linkage to CNT

### Take ______ ml of CNTs in SB and add to ______ ml of LB1

Mix at room temp for 15 hours.
Vacuum filter reaction with ______ um filter.
Wash CNT-GA with WB. Centrifuge at ______ g for ______ min.

### Remove Supernatant.

### Cross-Linking of Protein

Reconstitute CNT-GA with ______ ml of protein in LB2 at ______ mg/ml.
Mix for 1 hour at room temperature.
Filter CNT-GA-Protein molecule with ______ um filter.
Wash with WB. Centrifuge at ______ g for ______ min.
Storage? +4, −20, use right away?
Send for AFM analysis of binding.
Method 2—Adsorption of Proteins to CNT and then Glutaraldehyde Crosslinking.

### Solublization

Suspend CNTs in SB at a concentration of ______ mg/ml and sonicate for ______ hours.
Adsorption of Protein onto CNT-GA
Mix ______ mg of CNT in IB with ______ mg of protein in IB.
Mix at room temp for ______ hours.
Vacuum filter reaction with ______ um filter.
Wash with WB. Centrifuge at ______ g for ______ min.
Cross-Linking of Adsorbed Protein with Glutaraldehyde
Reconstitute CNT-GA/Protein with ______ ml of LB2.
Mix for 1 hour at room temperature.
Filter CNT-GA-Protein molecule with ______ um filter.
Wash with WB. Centrifuge at ______ g for ______ min.

### CNT Protocol (Sensor-Method 2)

I don't imagine method 1 will work well due to cross-linking of CNTs that aren't in the prescence of proteins

### Solublization

Suspend CNTs in SB at a concentration of ______ mg/ml and sonicate for ______ hours.

### Link to DSP on Sensor

Add 200 ul of CNTs in SB at ______mg/ml to each DSP modified well and incubate for ______ hours.
Wash sensor with SB.
Wash sensor with distilled water.
Wash sensor with LB1.
Incubate at room temperature for 15 hours.
Wash sensor with IB.
Make measurement.
Adsorption of Protein onto CNT-GA
Add 200 ul of protein at ______ mg/ml in IB to the sensor.
Incubate at room temperature for ______ hours.
Remove IB, but do not wash.
Cross-Linking of Adsorbed Protein with Glutaraldehyde
Add ______ of LB2 per well.
Incubate for ______ hours.
Wash with WB.
Make measurement

In one implementation, the system includes electrochemical sensors and arrays of such sensors with improved sensitivity to smaller amounts of, and more different kinds of analytes then is possible conventionally. The system may contain implementations of electrochemical sensor elements and circuits, strips and arrays of such sensors, delivery systems for transporting samples to be tested to the sensors or arrays, electrical and physical design of the electrochemical sensors. The relation of the electrical and physical design of the electrochemical sensors to the geospatial attributes, steric attributes, charge distribution attributes, electrical attributes, dielectric qualities, etc., and numerous other characteristics of the various analytes testable with the sensors is significant over conventional methods.

In one implementation, sensor arrays include partitioning the sensors for self-calibration, using a sensor to sense the uniformity or quality of its own manufacture, using part of a sensor surface as a calibration reference during manufacture for the remaining parts of the sensor surface, especially when the manufacture involves depositing a coating on the sensor, and especially when the coating is a nanoporous material selected for sensing one or more particular classes of analytes, or a particular individual analyte.

Multiple sensor elements can be gathered into one array, multiplexed or ganged in order to sense multiple analytes, contaminants, pesticides, biological compounds, trace materials, air constituents, gases, hormones, pharmaceuticals, heavy metals, minerals, food ingredients (and so forth) simultaneously. Individual sensor elements in an array can be turned on and off to tune the array toward a particular class of analyte or toward a particular individual contaminant. In one implementation, components of the sensor element and the detection circuitry are such that the sensor can detect an analyte in parts-per-billion (ppb).

In one implementation, an array of such sensors can process samples in real time at their native concentration, with no dilution or concentration of the samples needed. Thus, the sensors and arrays are immediately useful for water testing, air testing, food testing, contaminant testing, pharmaceutical testing, mining applications, chemical assays, biochemical assays, and so forth.

In one implementation, designs of the sensors are improved over conventional sensors because they may utilize a two wire or four wire design in combination with receptor-specific coatings to limit error in the signal detected and processed. A two-lead measurement technique may be used in some instances, in which the voltage is set and controlled across the lead and the current through the leads is measured. In the new sensor technology described below, a four lead measurement may also be employed, in which voltage is controlled across two leads by a feedback circuit and the current required to maintain the control voltage is measured on two separate leads. This results in less measurement uncertainty since any variation in series impedance of either the voltage or current leads has no effect on the measurement. This results in a new sensor electrical design for an electrochemical sensor that has significantly improved ability to measure with more precision and less error. This type of measurement has not previously been used on a potentiostat or similar environment.

There are many measurement applications in which the properties of a sensor are measured in order to determine a property of interest of a compound in contact with the sensor.

These measurements are referred to as indirect, in that the property of interest is inferred from the measurement of different property. For instance the concentration of an analyte may be inferred by the measurement of the capacitance of a sensor which is in contact with the analyte.

Of particular concern in this type of measurement is determining the relationship between the property that can be measured and the property of interest. In order to assert that a valid measurement has been made, it is necessary to demonstrate that an accurate and repeatable relationship exists between the two quantities.

In many applications, however, the relationship between the measured quantity and the quantity of interest is not easily established. One reason might be that the measurement sensor is consumed or destroyed in the measurement, meaning that it is not possible to perform a calibration step in addition to the measurement.

One such application is where the capacitance of a sensor is changed due to the chemical binding of an analyte to binding sites on the sensor. In this application, once the binding sites are occupied they are no longer available for further measurements. In this type of application it is necessary to have an accurate a priori understanding of how the sensor will react to the analyte. This understanding is often incomplete due to the many factors that might affect the sensitivity of the sensor during manufacture. For example, in the aforementioned capacitive sensors the current state of the art requires accurate control of concentration, volume and area of deposition of the active substance. This process is empirical in that once the sensor is complete only the nominal bulk property of the sensor is measured, even though many manufacturing processes may contribute to that property. The empirical nature of this process hampers statistical control of the manufacturing process and ultimately results in more measurement uncertainty when sensors are deployed in the field.

The purpose of this invention is to provide more resolution on the performance of a sensor in order to provide better control of the design, manufacturing and measurement process.

In one implementation, an example system addresses this goal by providing a means for dividing a single sensor into multiple parts which are separately measurable. Using this means during the development process, for example, allows direct measurement of the uniformity of the sensor's sensitivity when the various contributing processes are varied. During manufacture this means can be used to calibrate the sensor. When deployed in the field this means can be used to gain additional confidence in a measurement by providing multiple corroborating signals.

An example electrochemical sensor design and test device determines spatial orientation information about the substances to a sensor or an electrochemical sensor system. Each electrochemical sensor (hereinafter “sensor”) can be functionalized to adhere a specific class of chemical or substance, or a specific chemical or substance; or directed to a functional group, a molecular structure, molecular charge distribution, such as a specific protein, for instance. A particular sensor can be directed to a specific antibody, for example. Each sensor can be “tuned” to sensing an identity and/or concentration of an analyte by the physical layering of electrode elements, their dimensions, electrical and physical properties, by coatings such as nanoporous materials (e.g., nano carbon tubules) and activating agents.

In one implementation, the sensor elements are printed as conductors on a PC board, and modified to sense a certain chemical or biological species. Some of the illustrated PC boards have metal tabs adjacent to the sensors that are not needed if soldering an aluminum membrane to the sensor is not required.

An example system determines spatial orientation information about the binding of substances to a sensor or electrochemical sensor system to differentiate the substances from each other. The system achieves identification of species and higher sensitivity to concentrations via a two wire/four wire lead measuring design, in which the voltage is controlled across two leads by a feedback circuit and the current required to maintain the control voltage is measured on two separate leads.

The example system can provide a solution to the problems of determining: a) detection of substances by a sensor, b) binding of the substances to a sensor element or sensor system, c) adhesion or adherence of a substance to a sensor or sensing system. The system allows spatial and electrical information from the area over a sensor surface to be transmitted electronically and through electrochemical means. In one implementation, the system divides a single sensor or signal into multiple parts which are separately measurable. Using this partitioning during the development process or as quality control for manufacturing, allows direct measurement of the uniformity of the sensor's sensitivity when contributing processes are varied. The partitioning can also be used to calibrate the overall sensor. Calibration of each area of the sensor, since different patterns attributed to the manufacturing process can happen, allows for quality control in the manufacturing process, and when combined with overall sensor readings provides calibration for the overall system.

In a first case manufacturing scenario, a dosed blank is read a single time or multiple times by a sensor from each location over the sensor, and results are transmitted to the software. These readings are compared to known data and mathematical computations and can determine if each measure and the combined measure is within acceptable limits, and then establishes a corrected measure for each area and the sensor as a whole. For example, a test sensor can be incorporated into each panel of 100 sensors and data from a known dosed sample applied to the test sensor. Expected ranges from the dosed sample could be compared to the test sensor results to provide information about allowed variances that all other sensor on the board containing the test sensor can then be compared to as a means of determining defective sensors due to manufacturing.

In order to assert that a valid measurement has been made, it is necessary to demonstrate that an accurate and repeatable relationship exists between a quantity being measured in analogy for another quantity or identity. This information is especially important when trying to determine binding or functionalization of nanomaterials to a sensor or sensing system since nanotubes can be layered over a surface but not attached or functionalized in a manner to cause inaccurate readings because of a reduction of the sensor surface area. Additionally calibration can be difficult during manufacture when it cannot be determined if widely varying results are caused by other errors. The example system allows small areas to be compared to determine if areas on the sensor are left blank or nonfunctioning and the degree to which they vary. So, if a portion of the sensor is inadvertently left blank, for example, a correction for the blank section can be calculated and used for calibration or to determine if another sensor exceeded the blank range—so that some other problem must exist or whether, on the other hand, the overall sensor can still be standardized based on tolerances or parameter limits.

In many applications, however, the relationship between the measured quantity and the quantity of interest is not so easily established. One reason might be that the measurement sensor is consumed or destroyed in the measurement, meaning that it is not possible to perform a calibration step in addition to the measurement.

Once such an application is where the capacitance of a sensor is changed due to chemical binding of an analyte to binding sites on the sensor. In such an application, once the binding sites are no longer available for further measurements. In this type of application it is necessary to have an accurate and prior understanding of how the sensor reacts to or with the analyte. This understanding is often incomplete due to many factors, including those caused by manufacturing processes that affect the sensitivity of the sensor. For example, in the aforementioned capacitive sensors, the current state of the art requires accurate control of concentration, volume and area of deposition of the active substance. This process is empirical in that once that the sensor is complete only the nominal bulk property of the sensor is measured, even though many manufacturing processes may contribute to that property. The empirical nature hampers statistical control of the manufacturing process and ultimately results in more measurement uncertainty when sensors are deployed in the field.

Thus, the example system can provide more resolution on the performance of a sensor in order to provide better control of the design, manufacture, and resulting measurement processes.

Manufacturing sensors composed of nano or micro components attached to an electrochemical sensor base of substrates such as PCB, silicon chip, or other substance is currently unreliable because no effective means of assessing the quantity and functionalization of the nanoporous membranes exists to determine such issues as adhesion of a substance or an agent to the sensor, the number of functionalized pores, and uniformity of functionalized surface. The example system, however, solves many of these problems by measuring sections included in one sensor electronically to determine the total capacitance of each small section constituting the overall sensor and comparing the measurement with other sections to determine total capacitance. This partitioned surface information allows the manufacturer to determine if the microporous material is uniformly distributed and functionalized over the sensor. It also allows the manufacturer to determine if the manufacturing process of the attachment of nanotubes or other nanoporous material in the sensor system is reliable and repeatable. This system additionally provides a robust method of calibration. For calibration, the distribution of multiple signals coming from an overall sensor can be compared to a construct generated from one section.

A partitioned sensor can establish a baseline sensor functionality to determine manufacturing inconsistencies and correct for them. Similarly, other factors can be assessed such as: 1) adhesion force of the nanoporous membrane to the sensor, 2) uniformity of functionalization of the nanoporous membrane over the sensor surface and 3) uniformity and amount of binding of antibodies or other materials to the membrane.

The example system may enable multiple measurements under the umbrella of one sensor, which are measured to allow sensor surface information to be collected across the area surface of the sensor. This can generate many types of testing information such as how the rate or flow of fluid over the sensor helps or hinders attachment of the target material to the sensor. This new and unique design affords geospatially or surface-section-specific descriptive information for manufacturing processes and for calibration, yielding an increase in accuracy and understanding of attachment and dispersion of nanoporous surfaces over a sensor, functionalization of nanoporous surfaces, and binding of species to nonporous functionalized surfaces—as well as fluid or sample delivery control issues and how they impact binding and adhesion.

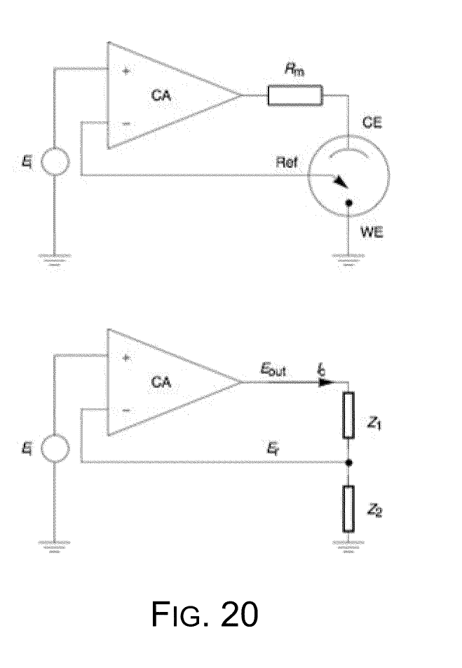

A range of sensor designs are presented to demonstrate the use of a PCB based or other material based sensor for electrochemical detection of antibodies, proteins or other substances such as pesticides, heavy metals, pharmaceutical drugs, bacteria and biological compounds of interest. In this embodiment, the design of the sensor array utilizes a two or four wire design to limit error in the signal. As shown in FIG. 20, a potentiostat may be utilized for electrochemical measurement. A two-lead measurement technique may have the voltage is set, i.e., fixed, and controlled—maintained to be constant—across the lead and the current through the two leads is measured. In one implementation of the example system, a four lead measurement schema causes the voltage to be controlled across two leads by a feedback circuit while the current required to maintain the control voltage is measured on two separate leads. This results in less measurement uncertainty since any variation in series impedance of either the voltage or current leads has no effect on the measurement. This results in a new sensor electrical design for an electrochemical sensor that has significantly improved ability to measure with more precision and less error. This type of measurement has not previously been used on a potentiostat or similar measuring instrument.

For example in one embodiment the ECM (Electrochemical Monitoring System) can employ 10 to 80 electrochemical sensors and controls the connection of sensor to a potentiostat or to embedded electrical hardware for signal generation and analysis through individual units or multiplexing. The ECM 300 may also control valves and pumps incorporated in the unit for sample and fluid delivery to each individual electrochemical sensor. The electrochemical sensors are designed with two or three electrodes of immersion metal (gold, silver, platinum, copper or other metal or conductive material) with each electrode having two or three electrical connections adhering to a four wire resistor model so that in one embodiment the sensor is composed of a four wire lead design for measuring where the voltage is controlled across two leads by a feedback circuit and the current required to maintain the control voltage is measured on two separate leads. In this example a gold immersion electrode is used. The model may be the following or a variation of the following model:

[Z=Rp(SRs Cx+1)]/[S2(Cp Cx Rs Rp)+S(Cp Rp+Cx Rs+Cx Rp)+1]

For:

Rp>>1; Rs<<1; and Cp<<Cx.

The device may contains circuitry providing functionality of a potentiostat enabling a portable device. Another embodiment of the device enables multiplexing to a potentiostat.

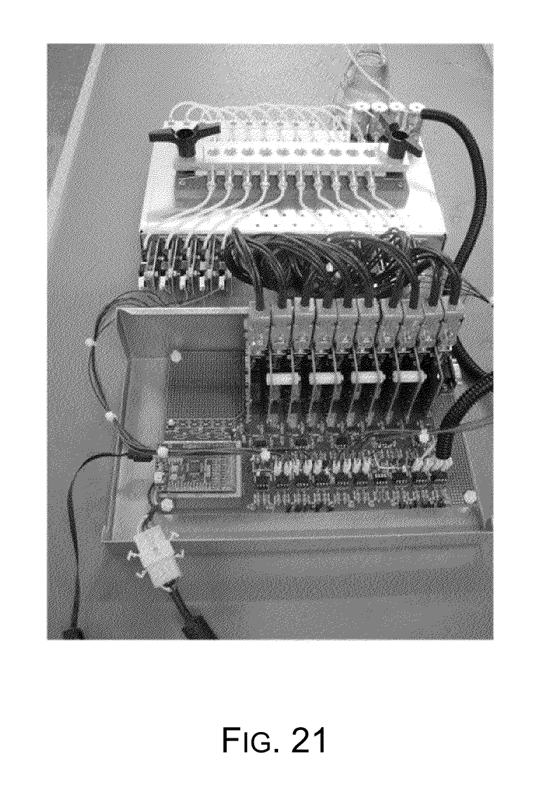

An example system may include a multiplexing fixture (platform) (consisting of an array of sensors, each having sensor elements, a fluid handling system for the samples to be texted in multiple cells though a manifold, and electrical platform, a software platform for control, multiplexor hardware, and measurement instrumentation typically consisting of a potentiostat and related circuitry, e.g., for electrochemical impedance spectroscopy. Wiring to the sensors can be through dedicated cable sets soldered to the sensor boards or electrical contact may be made through a spring bed (“bed of nails”). The multiplexer can be stepped through the channels manually or automatically. FIG. 21 shows an example system, having ten multiplexor daughter boards connected to a mother board with and controlled with a pick; ten piezoelectric pumps connected to ten daughter boards then connected to a pick to control the fluid delivered to and from the sensor with four valves connected to a pick to control choice of fluids to the sensor. Some sensor styles are shown in FIGS. 1-4, while the multiplexer can be an ECM300 or equivalent feeding a four wire sensing schema to a Gamry potentiostat.

Twenty sensor strips and manifolds of the selected design can be deployed, in one implementation. Up to six full panel concentration runs can currently be done on each of multiple different analytes.

Exemplary Potentiostats

Physical electrochemistry experiments routinely use small electrodes, so currents tend to be low. Gamry Potentiostats are particularly well-suited for low current measurement because of the low noise inherent in their design. Depending on implementation, the Series G 300 Potentiostat or the Reference 600 Potentiostat can be used. A handheld model may include smaller impedance spectroscopy hardware and firmware. A mobile sensor array may also be implemented with a Gamry Reference 600 Potentiostat and notebook computer, for example. An example system may use a Randles or Warburg type circuit with Warburg element, with ground and counter/reference/working electrodes. The design provides very low noise. An EIS spectrum can be shown in a Nyquist plot. The four wire design minimizes conversion surface charge to capacitance (i.e., noise). A 3-circuit design may also be used. The potentiostat system can be configured to scan for the e-signatures of various species and substances and can also sense changes in impedance according to how much material is on given sensor. An aggregate view determines how much of each material is on all sensors of an array, and can compare against one or more calibration sensors.

Example software may include PHE200 physical electrochemistry software for cyclic voltammetry, linear sweep voltammetry, chronoamperometry, chronocoulometry, chronopotentiometry, and controlled potential coulometry. The PHE200 also includes multiple-step chronoamperometry and chronopotentiometry techniques that can be useful for a multiplexed array of the example sensors.

PV220 Pulse voltammetry software can be used to implement square wave, differential pulse, normal pulse, reverse normal pulse, and sampled DC voltammetry. A custom generic pulse generator provides pre-defined potentiostatic or galvanostatic pulse waveforms.

Physical electrochemistry addresses the broad area of fundamental electrochemistry. This includes theoretical and experimental aspects of double-layer structure, kinetic and mechanistic studies of heterogeneous electron transfer at electrode-electrolyte interfaces, electrocatalysis, and the application of spectroscopic and other techniques to the study of electrochemical interfaces and processes.

Cyclic voltammetry can be tuned to the kinetics of the electrochemical reaction by adjusting the scan rate. Other techniques commonly used for research electrochemistry include electrochemical impedance spectroscopy, chronoamperometry, chronocoulometry, and chronopotentiometry.

Electroanalytical chemistry is a complementary niche of physical electrochemistry. There are a wide range of “pulse techniques” that are often employed by electroanalytical chemists. These techniques include differential pulse, square wave, normal pulse, reverse normal pulse. The pulse techniques are rarely used for fundamental electrochemical studies. But because they are incredibly sensitive compared to cyclic voltammetry, they can be handy to have in your electrochemical tool kit. The pulse techniques can also be used in stripping voltammetry in which a solution species is electrochemically pre-concentrated onto an electrode surface, then quantitated using the pulse waveform.

In one implementation, a particular sensor utilizes steric and electronic properties of the target analytes to identify them. In one implementation, heterogeneous integration of a microelectronic platform with nanomaterials forms nanoscale confined spaces, which substantially match the size of analytes detected on the sensor surface. In one implementation, specific chemicals, when interacting with the nanomaterial, produce variations in electrical parameters, such as current, voltage, and impedance, which are integrated to generate an electrical signature. An exemplary technique enables multiplex detection of multiple analytes using a single sensor device by probing the frequency response of the analyte mixture and comparing frequency responses or signatures with a library of frequency response signatures of individual analytes that compose the mixture.

In one implementation of the electrochemical sensor, a non-invasive electrical monitoring system selectively distinguishes and detects chemicals for example pesticides, pathogens, and a wide range of analytes that can be classified as small molecules in a non-invasive manner. Detection of small molecules is achieved through receptor-based binding onto the sensor surface. The quality of water, whether it is used for drinking, irrigation or recreational purposes, is significant for health. One implementation of the electrochemical sensor is that it non-invasively detects pesticides that degrade the quality of water and produce adverse health effects when pesticide concentrations are in the range of picograms/ml. In one illustrative example, a representative pesticide binding system detects three specific pesticides: Endosulfan sulfate, Edrin aldehyde, and Edrin ketone.

An example pesticide binding system uses an improved surface-area-to-volume ratio as well as multi-scale architecture while interfacing functional nanomaterials with microelectronic circuits toward building a non-invasive, electrochemical sensor, that immediately responds to the binding of specific pesticides on the nanotextured surfaces. Robustness and selectivity can be achieved through functionalization of multiwalled carbon nanotubes and a controlled sheet-based deposition onto microelectronic circuits.

Chemical Binding System

In one implementation, a pesticide binding system isolates and immobilizes the pesticides from fluid media and onto measurement sites. This same principal may be used for a number of different applications to measure pesticides, heavy metals, pharmaceutical drugs, bacteria, or many other biochemical targets in water, bodily fluids, foods and soil. As shown in the Figures, example devices may consist of a base microelectrode platform comprising multiple instances and styles of sensor elements, e.g., as fabricated using standard photolithography techniques of masking, patterning, developing, metal deposition and lift off. The microelectrode platform may be encompassed by a polymeric encapsulant for controlling fluid flow into and out of the platform. The flow rate can be controlled by a micro or syringe pump (Harvard Instruments, Kent Scientific). The metal micro electrodes can be made of platinum or other metals; for example, 80 μm in diameter with a 200 μm center-to-center spacing. Each microelectrode is connected to a rectangular measurement pad (e.g., 200 μm×240 μm) through an electrode lead, e.g., 10 μm in width and approximately 750 μm in length.

Multi-walled semi conducting nanotubes (MWCNTs) can be used as transporters to bind the chemical analytes, such as pesticides, from sample solutions. In one implementation, these carbon nanotubes are functionalized with receptors for Endosulfan sulfate, Edrin aldehyde, Edrin ketone respectively. In one case, these are monoclonal antibodies in Tris buffer with CTAB as a surfactant. Hence, in one example, three carbon nanotube samples functionalized with the three receptors are generated. The MWCNTs are first functionalized with covalent amine linkers. The stock solution of MWCNTs is then aliquoted into, e.g., three separate stocks. Each sample of the re-dispensed stock is comprised of the amine functionalized MWCNTs. These MWCNTs are then incubated in chemical cocktail comprising the antibody and the buffer. This ensures saturation of the MWCNTs with the antibody. In one example, the relevant antibody functionalized MWCNTs are then incubated for 5 minutes with the test sample. This incubation ensures binding of the relevant pesticide onto the functionalized carbon nanotube surfaces.

Electrical Detection of Pesticides

The pesticides bound to the MWCNTs can be separated from the unbound MWCNTs using gradient electric fields also known as alternating current fields. The application of gradient electric fields produces a dipole in the cluster of MWCNTs. Due to the different dielectric properties of the pesticide bound MWCNTs from the unbound MWCNTs the intrinsic polarizability of these two types of MWCNTs changes for a specific applied electric field. This in turn separates the pesticides. The geometry as well as the locations of the application of the field is determined through mathematical multiphysics field-based models. By empirically varying the applied peak-to-peak voltage and the frequency of the applied field, the polarizability of the pesticide bound MWCNTs are modulated such that they get collected on the measurement site allowing for their rapid and continuous monitoring. Once the pesticide bound MWCNts are separated onto the metallic measurement sites, the electric field is turned off. Due to the intrinsic inertia as well as the trapping process of the MWCNTs, they remain localized onto the measurement sites from approximately two to five minutes allowing for a measurement from the microelectrode sites. In one implementation, the gradient field is turned ON and OFF with an ON time of, for example, two minutes and an OFF time varying, for example, from two to five minutes.

Example Apparatus

FIG. 21, as noted above, shows an example apparatus that operates with the described electrochemical sensor.

The design of the sensor is superior to other sensor designs in that it allows the size of the sensor to be scaled up or down without reduction in the range in which the device can measurement with precision. In fact the design enables measurements 100× lower than parts-per-billion. In one implementation, an electronically controlled device that holds the sensor is uniquely designed to limit interference and precisely control fluid delivery to the sensor so that fluid does not come in contact with the electrical components used to generate and measure electrical signals. This further enables validity, reliability and precision in measurement. The electrical and chemical design of the sensor allows specificity of measurement. Example: measurement of specific pesticides, etc., is enabled, not just measurement of general groups.

The design of the test fixture allows any sensor, chip or board to be tested with minimal adjustment of the fixture based on the size and functionality of the sensor, chip, or board by use of an adjustable insert which holds the test strip, chip, sensor or board. This robust design allows researchers a fixture which is fully flexible for type of product being tested and also for how fluid, electrical impulses and signals are controlled. The example design provides a unique benefit to researchers and in experimental development has decreased training time for graduate students by 50% and has increased the number of tests that can be performed in a day by 100%, e.g., from 5 per day to 10 per day. This effectively reduces testing time by 50%. This is a significant increase and increases the depth and breadth of discovery and innovation per research dollar spent.

The example design can be automated and adapted to enable remote automated testing of water, soil or food. In the shown embodiment, the device can be used 1) on a food processing line, where it can send data on the measurement of different substances such as pesticides or heavy metals to a database for analysis, or can be used to print tags stating the level of measured pesticide, heavy metal or other substance found in the food, to be attached to the actual food item (i.e., fish fillet); 2) as an automated remote monitoring device submerged in water or located near water in which sampling rate, time and date can be programmed into the device by computer via a web or manual interface and the results delivered to a database for access by computer. The device may also be loaded with enough sensors, boards, and chips to enable measurement multiple times per day for a year or more, unattended.

A novel delivery and activation method delivers antibody, reagent or other solution to the sensor and provides delayed activation for later use. An applier, a device similar to an ink-jet printer, deposits antibodies or chemicals on the chip, which can be later hydrated to activate them. This allows an extended shelf life of numerous substances and reduces the need for replacement of the device or its servicing while preventing traditional liquid fluids from degradation. This enables long-term remote monitoring since traditional methods require these antibodies or other chemicals to be mixed and activated within a few minutes or a few days of use to be viable for the use.

In one implementation, the design of the device may utilize solar collectors and/or mini water turbines to provide renewable power to the device and keep batteries charged. This allows the device to be self-sustainable in the field with no maintenance. The device can use a holding membrane which allows sand or other materials to settle out, then water can be pumped from the upper portion and agitated to provide a representative sample. This technique enables field sampling without use of a filter. Pressurized air followed, as needed, by distilled water may be used to clear the system. Ultrasound or microwaves can be used as an antibacterial method for the device. Various pneumatic and electrical feedback systems provide the user with information about mechanical and electrical functioning and malfunctioning of the device. A handheld version of the device may follow the same design but incorporate additional automation. In one implementation, a laser may be incorporated into the device to vaporize samples for measurement.

Example Method

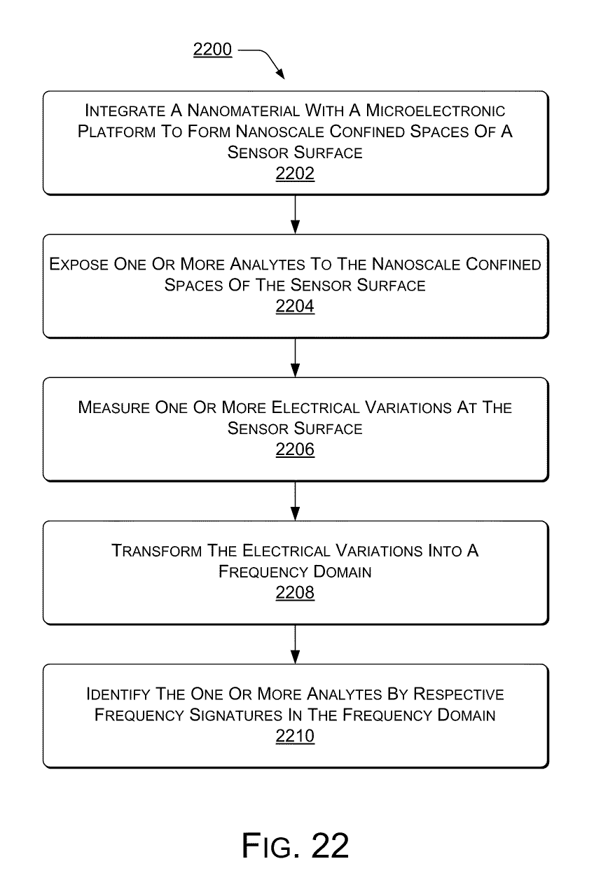

FIG. 22 is a flow diagram of an example method of identifying a chemical at a nanoscale confined space of a electrochemical sensor surface via an electronic signature. In the flow diagram, the operations are summarized in individual blocks. The example method 800 may be performed by hardware such an exemplary electrochemical sensor.

At block 2202, a microelectronic nanomaterial is heterogeneously integrated with a microelectronic platform to form nanoscale confined spaces of a sensor surface.

At block 2204, multiple analytes are exposed to the nanoscale confined spaces of the sensor surface.

At block 2206, one or more electrical variations are measured at the sensor surface.

At block 2208, the electrical variations are transformed into a frequency domain.

At block 2210, the one or more analytes are identified by respective frequency signatures in the frequency domain.

1. An electrochemical sensor system for measuring contaminants, comprising:
a sensor including two conductors arranged in a capacitive relationship on a printed circuit board;
a binding layer attached to at least one of the conductors to selectively bind a particular contaminant to the binding layer;
a sensing module connected to the two conductors to form an electrical circuit and measure a change in impedance resulting from an amount of the particular contaminant currently bound to the binding layer; and
an enclosure around at least part of the two conductors to maintain an environment of the particular contaminant for real time measurement of the particular contaminant.

2. The electrochemical sensor system of claim 1, wherein the capacitive relationship of the two conductors is tuned to the particular contaminant.

3. The electrochemical sensor system of claim 2, wherein the capacitive relationship is tuned to a particular contaminant by varying one of:
a pattern of the two conductors printed on the printed circuit board;
a configuration of the two conductors printed on the printed circuit board;
a physical characteristic of a material composing at least one of the two conductors;
a doping of at least one of the two conductors;
a length of at least one of the two conductors;
a width of at least one of the two conductors on the printed circuit board;
a distance between the two conductors on the printed circuit board; or
an overall length of the two conductors disposed at a given distance from each other.

4. The electrochemical sensor system of claim 1, wherein a size of a surface area of a pattern of the two conductors printed on the printed circuit board is selected to provide a sensitivity range for sensing the particular contaminant.

5. The electrochemical sensor system of claim 1, wherein the binding layer is tuned or selected to bind or ligate to the particular contaminant.

6. The electrochemical sensor system of claim 1, wherein the sensing module is configured to measure an impedance indicative of dielectric changes in the binding layer caused by an amount of the particular contaminant currently bound to the binding layer on at least one of the two conductors.

7. The electrochemical sensor system of claim 1, wherein the sensing module is configured to measure an impedance based on electrical or charge properties of a chemical bilayer formed by an amount of the particular contaminant currently bound to the binding layer on at least one of the two conductors.

8. The electrochemical sensor system of claim 1, further comprising a nanoporous layer between a conducting surface of at least one of the two conductors and the corresponding binding layer to increase a detection sensitivity of the sensor; and
wherein the nanoporous layer comprises one of an alumina membrane, a carbon nanotube (CNT) membrane, or a multi-wall carbon nanotube (MWCT) membrane.

9. The electrochemical sensor system of claim 8, wherein the nanoporous layer utilizes steric and electronic properties of the particular contaminant to identify the contaminant.

10. The electrochemical sensor system of claim 1, wherein the sensing module performs multiplex detection of multiple contaminants by probing a frequency response of a contaminant mixture and comparing obtained frequency responses against a library of frequency response signatures of individual contaminants.

11. The electrochemical sensor system of claim 1, further comprising an array of the sensors, each sensor in the array tuned to a different contaminant.

12. The electrochemical sensor system of claim 11, wherein each sensor in the array of sensors is multiplexed to an array of potentiostats to detect multiple contaminants simultaneously, in real time.

13. The electrochemical sensor system of claim 11, further comprising a delivery system to automatically partition a sample containing contaminants into multiple parts and deliver the multiple parts to the corresponding enclosure for each sensor in the array of sensors.

14. The electrochemical sensor system of claim 13, further comprising one or more buffer solutions for automatic deliver to the corresponding enclosure for each sensor in the array of sensors, the one or more buffer solutions selected from the list of buffer solutions including:
a solubilization buffer;
an immobilization buffer;
a wash buffer; and
a linking buffer; and
wherein the one or more buffer solutions maintain the particular contaminant in solution as a correct species or chemical form of the contaminant for binding with the binding layer.

15. The electrochemical sensor system of claim 11, wherein the different contaminants to be sensed by the array of sensors include pesticides, herbicides, proteins, heavy metals, antibodies, drugs, and bacteria:
wherein the pesticides include malathion and chlorpyrifos;
wherein the herbicides include atrazine; and
wherein the heavy metals include mercury and lead.

16. The electrochemical sensor system of claim 1, wherein the binding layer comprises one of:
a mercury binding protein;
a heavy metal binding protein;
an anti-atrazine antibody;
an anti-malathion antibody;
an anti-chlorpyrifos antibody;
an enzyme;
an oligosaccharide;
a nucleotide or oligonucleotide;
a cholinesterase enzyme;
a pesticide binding protein;
a metallo-enzyme;
a cell receptor protein;
a peptide;
a lipid;
a protein;
a drug;
a drug target;
a neurotransmitter;
a carboxyl group;
a herbicide binding protein;
a fungicide binding protein;
a biomarker binding protein;
a DNA binding protein; or
a DNA motif.

17. The electrochemical sensor system of claim 1, further comprising a pulse generator to send an electrical pulse across the two conductors;
wherein the electrical pulse comprises a voltage waveform or a current waveform; and
wherein the electrical pulse is tailored to detect an impedance signature of a specific contaminant.

18. The electrochemical sensor system of claim 1, wherein the binding layer binds the particular contaminant reversibly and the monitor provides ongoing real time measurement of a dynamic concentration of the particular contaminant in one of a gas, a liquid, air, or a water sample.

19. The electrochemical sensor system of claim 18, wherein an array of the sensors, each sensor tuned to a different contaminant, provide ongoing real time measurement of dynamic concentrations of multiple contaminants in one of a gas, a liquid, air, or a water sample.

20. The electrochemical sensor system of claim 18, further comprising a laser processing unit for sample preparation.

21. The electrochemical sensor system of claim 18, further comprising a sampling system configured to draw a sample directly from a food processing line and/or process the sample by dilution in fluid, dissolution in fluid, digestion, bead blundering, and/or vaporization and dissolution in fluid, and transfer to the fluid delivery system for deposition on the sensor.

22. The electrochemical sensor system of claim 21, further comprising software and hardware programmed to take analog information from the sensing module and translate the information to digital data for storage in a database; and
software and hardware to print the name and amount of contaminants found in the food sample on a tag, barcode, or label to be attached or printed on a food product.
